(19) United States
(12) Patent Application Publication
(10) Pub. No.: US 2026/0334844 A1
(43) Pub. Date: Oct. 29, 2026

(54) ESTIMATION PROGRAM, ESTIMATION DEVICE, AND METHOD

(71) Applicant: KEIO UNIVERSITY, Tokyo (JP)

(72) Inventors: Yoshinori KATSUMATA, Tokyo (JP); Shinichi GOTO, Kanagawa (JP); Kotaro MIURA, Tokyo (JP)

(21) Appl. No.: 19/471,866

(22) PCT Filed: Apr. 1, 2024
(86) PCT No.: PCT/JP2024/013526

§ 371 Date: Oct. 2, 2025

(30) Foreign Application Priority Data
Apr. 3, 2023 (JP) 2023-060378

(51) Int. Cl. A61B 5/346; A61B 5/282; A61B 5/339

(52) U.S. Cl. CPC A61B 5/346; A61B 5/282; A61B 5/339

(57) ABSTRACT

Assists in the diagnosis of atrial septal defect. The program according to an embodiment of the present invention ins a program for causing an estimation device to function as: an electrocardiogram (ECG) acquisition unit configured to acquire an ECG of a patient; a potential data acquisition unit configured to acquire time-series data of a potential underlying the ECG of the patient from the ECG of the patient; an estimation unit configured to input the time-series data of the potential underlying the ECG of the patient into an estimation model that estimates a probability that a subject has an atrial septal defect based on time-series data of a potential underlying an ECG of the subject, and output a probability that the patient has the atrial septal defect; and a display unit configured to display the probability that the patient has the atrial septal defect.

### TECHNICAL FIELD

The present invention relates to an estimation program, an estimation device, and a method.

### BACKGROUND ART

In conventional medical practice, complex data such as electrocardiograms, still images, and moving images are used. In normal clinical settings, humans (physicians) review this complex data, read the findings, and make clinical judgments. Correctly interpreting such complex data requires extensive training.

Recently, Artificial Intelligence (AI) technology using neural networks has advanced. In the medical field, attempts are being made to directly train AI on complex data to extract clinically useful information. In many medical fields, significant successes have been achieved, such as making estimations with accuracy surpassing that of physicians and accomplishing tasks previously impossible. AI using neural networks can automatically extract features from data and compute results without being explicitly taught how humans evaluate things, provided it learns a large number of input data points and corresponding correct answers. Consequently, it has an extremely wide range of applications and holds the potential to extract information that humans may not even recognize.

ECGs, in particular, are complex data containing abundant information despite being easy to acquire and providing immediate results. Consequently, they represent a field where numerous successes have been achieved through AI application (Non-Patent Document 1).

### PRIOR ART DOCUMENTS

### Non-Patent Documents

Non-Patent Document 1: H. Mori et al., Pediatric Cardiology (2021) 42:1379-1387 10.1007/s00246-021-02622-0

### DISCLOSURE OF THE INVENTION

### Problem to be Solved by the Invention

In the field of cardiology, several diseases exist that progress asymptomatically yet can lead to serious conditions such as future heart failure or cryptogenic cerebral infarction. Many of these could be detected and treated if suspected and examined, but they are currently being missed. Screening for such diseases on a large scale using simple tests like electrocardiograms would be of significant importance.

Atrial septal defect (ASD) is one such disease. It is the most common congenital heart defect, occurring in approximately 0.5% of all births. However, many cases remain asymptomatic and thus go undetected. Conversely, it increases the risk of stroke, atrial fibrillation, and heart failure, sometimes leading to ASD being discovered when these conditions present as the initial symptom. However, these complications are irreversible, and closing the atrial septal defect at this stage is too late. Early detection allows for relatively minimally invasive closure of the defect using a catheter, making it essential to efficiently identify ASD in asymptomatic cases.

Therefore, the present invention aims to assist in the diagnosis of atrial septal defect.

### Means of Solving the Problem

A program according to an embodiment of the present invention provides an estimation device comprising: an ECG acquisition unit for acquiring a patient's electrocardiogram; a potential data acquisition unit that acquires time-series data of the underlying electrical potentials of the patient's ECG from the patient's ECG, an estimation unit that inputs the time-series data of the underlying electrical potentials of the patient's ECG into an estimation model that estimates the probability that the subject has atrial septal defect from the time-series data of the underlying electrical potentials of the subject's ECG, and outputs the probability that the patient has atrial septal defect, and a display unit that displays the probability that the patient has atrial septal defect.

### Advantageous Effects of Invention

According to the present invention, diagnosis of atrial septal defect can be supported.

### BRIEF DESCRIPTION OF THE DRAWINGS

FIG. 1 is a diagram showing the overall configuration of an embodiment of the present invention.

FIG. 2 is a functional block diagram of a learning device according to an embodiment of the present invention.

FIG. 3 is a diagram for explaining the time-series data of the original potential of the electrocardiogram according to an embodiment of the present invention.

FIG. 4 is a diagram illustrating the estimation model according to an embodiment of the present invention.

FIG. 5 is a functional block diagram of an electrocardiogram according to an embodiment of the present invention (for Example 1).

FIG. 6 is a functional block diagram of an ECG management device according to an embodiment of the present invention (for Example 2 and Example 3).

FIG. 7 is a flowchart of the learning process according to an embodiment of the present invention.

FIG. 8 is a flowchart of the estimation process according to an embodiment of the present invention.

FIG. 9 is an example of displaying an ECG and the probability that a patient has atrial septal defect according to an embodiment of the present invention.

FIG. 10 is a diagram illustrating the accuracy of the estimation model according to an embodiment of the present invention.

FIG. 11 is a hardware configuration diagram of an electrocardiograph according to an embodiment of the present invention.

FIG. 12 is a hardware configuration diagram of the learning device and ECG management device according to an embodiment of the present invention.

### MODE OF CARRYING OUT THE INVENTION

The embodiments of the invention will now be described with reference to the drawings.

### Atrial Septal Defect (ASD)

First, atrial septal defect (also known as ASD) is explained. Atrial septal defect is a congenital heart malformation and is the most common congenital heart disease. Many cases progress asymptomatically, and numerous cases remain undetected until adulthood.

However, atrial septal defect carries the risk of shunting venous blood into arterial blood, potentially causing various cardiovascular diseases such as increased incidence of cerebral infarction and heart failure. Early detection, evaluation of hemodynamics, and appropriate treatment are desirable. Currently, detecting atrial septal defect requires imaging tests such as echocardiography for suspected cases based on symptoms or auscultation findings. These imaging tests are problematic due to their labor-intensive nature, radiation exposure, and high cost, making large-scale screening implementation impractical. Consequently, asymptomatic cases without clear findings suggestive of atrial septal defect are often missed, necessitating an effective screening method.

### System Configuration

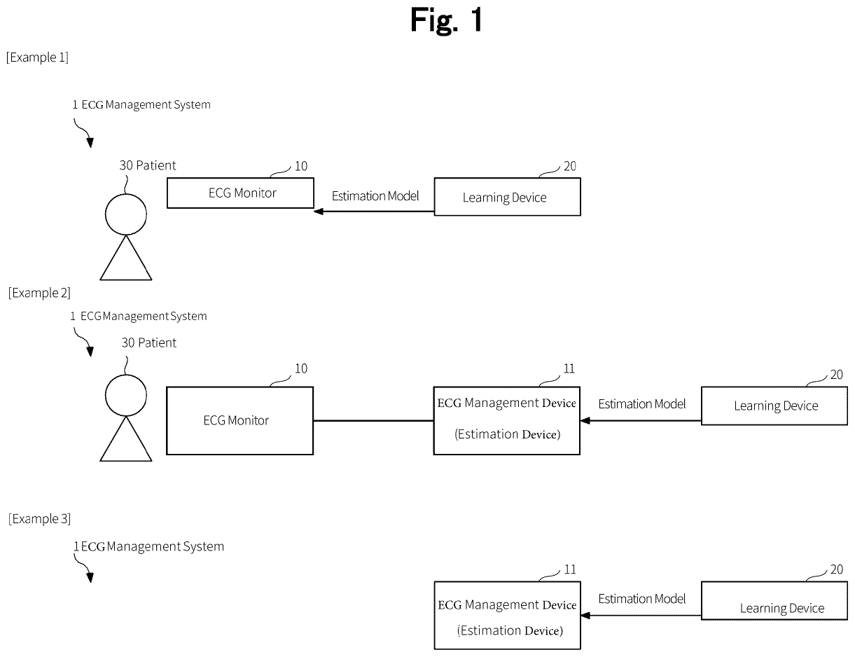

FIG. 1 is a diagram showing the overall configuration of an embodiment of the present invention. It will be explained in Examples 1, 2, and 3. In Example 1, an electrocardiograph 10 estimates the probability that patient 30 has atrial septal defect. In Examples 2 and 3, an ECG management device 11 (e.g., a computer such as a personal computer or server) capable of acquiring ECG potential data estimates the probability that patient 30 has atrial septal defect. In this specification, patient 30 refers to an individual undergoing examination by a physician at a medical institution, undergoing a health checkup, etc.

### Example 1

The ECG management system 1 includes an ECG monitor 10.

### <<Electrocardiograph>>

The electrocardiograph (an example of an estimation device) 10 measures the electrocardiogram of the patient 30. The ECG monitor 10 inputs the time-series data of the underlying electrical potentials (also referred to as ECG potential data) of the subject's ECG into an estimation model that estimates the probability that the subject has atrial septal defect. outputs the probability that the patient 30 has an atrial septal defect, and displays the probability that the patient 30 has an atrial septal defect. Note that the ECG monitor 10 is assumed to possess the estimation model generated by the learning device 20.

### Example 2

The ECG management system 1 includes an ECG monitor 10 and an ECG management device 11. The ECG monitor 10 and the ECG management device 11 may be connected via any network (wired or wireless) for communication, or they may be connected by a cable or the like.

### <<ECG Monitor>>

The ECG monitor 10 measures the ECG of the patient 30. The ECG monitor 10 transmits the measured ECG to the ECG management device 11, and the ECG management device 11 receives the measured ECG from the ECG monitor 10.

### <<ECG Management Device>>

The ECG management device (an example of an estimation device) 11 comprises one or more computers. The ECG management device 11 inputs the time-series data of the underlying electrical potentials (ECG potential data) of the subject's ECG into an estimation model that estimates the probability that the subject has atrial septal defect. outputs the probability that the patient 30 has an atrial septal defect, and displays the probability that the patient 30 has an atrial septal defect (this may be displayed on the ECG management device 11 or on the ECG monitor 10). Note that the ECG management device 11 is assumed to possess the estimation model generated by the learning device 20.

The ECG management device 11 may also acquire the patient 30's ECG measured by the ECG monitor 10 in real time and estimate the probability that the patient 30 has an atrial septal defect in real time. Alternatively, the ECG management device 11 may acquire the patient 30's ECG previously measured by the ECG monitor 10 and estimate the probability that the patient 30 has atrial septal defect.

### Example 3

The ECG management system 1 includes an ECG management device 11. The ECG management device 11 is capable of acquiring ECGs measured by any ECG monitor (i.e., ECGs measured at any medical institution).

### <<ECG Management Device>>

The ECG management device (an example of an estimation device) 11 comprises one or more computers. The ECG management device 11 inputs the time-series data of the underlying electrical potentials of the subject's ECG (ECG potential data) into an estimation model that estimates the probability that the subject has atrial septal defect. outputs the probability that the patient 30 has atrial septal defect, and displays the probability that the patient 30 has atrial septal defect (which may be displayed on the ECG management device 11 or on another terminal). Note that the ECG management device 11 is assumed to possess the estimation model generated by the learning device 20.

### Functional Blocks

The functional configuration is described below.

### [Learning Device]

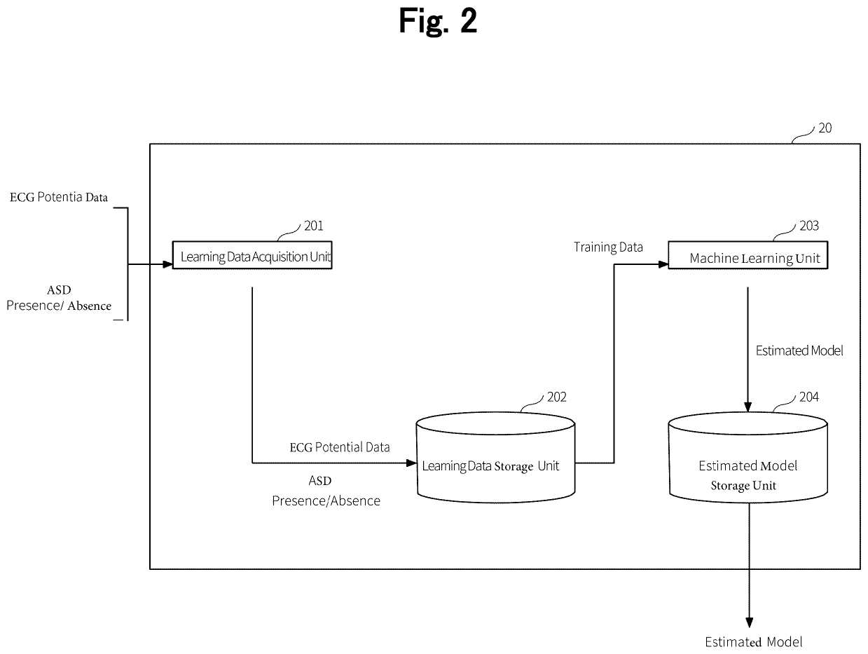

FIG. 2 is a functional block diagram of the learning device 20 according to an embodiment of the present invention. The learning device 20 may include a learning data acquisition unit 201, a learning data storage unit 202, a machine learning unit 203, and an estimation model storage unit 204. The learning device 20 can function as the learning data acquisition unit 201 and the machine learning unit 203 by executing a program. The following describes each of these units.

The learning data acquisition unit 201 acquires learning data used to generate an estimation model. Specifically, the learning data acquisition unit 201 acquires paired data consisting of time-series data of the electrical potential underlying the subject's electrocardiogram (ECG potential data) and information indicating whether or not the subject has atrial septal defect.

### [ECG Potential Data]

The subject's ECG is, for example, a 12-lead ECG. A 12-lead ECG uses four limb leads attached to the limbs and six chest leads attached to the chest. Six waveforms are derived from the limb leads, and one waveform is derived from each of the chest leads. Therefore, a total of twelve waveforms (I, II, III, aVR, aVL, aVF, V1, V2, V3, V4, V5, V6) are recorded. As learning data, a two-dimensional matrix is used, where the vertical axis represents each lead (each of the 12 leads) and the horizontal axis represents time (i.e., a matrix showing the voltage values at each time point for each lead).

### [Information Indicating Presence or Absence of Atrial Septal Defect]

Information indicating the presence or absence of atrial septal defect is the result of a physician's diagnosis of whether the subject has atrial septal defect.

The learning data storage unit 202 stores the subject's ECG potential data (specifically, a two-dimensional matrix where the vertical axis represents each lead (each of the 12 leads) and the horizontal axis represents time, i.e., a matrix showing the potential values at each time point for each lead) and information indicating whether the subject has an atrial septal defect.

The machine learning unit 203 generates an estimation model through machine learning. Specifically, the machine learning unit 203 adjusts the parameters of the estimation model to minimize the error between the information indicating whether the subject has an atrial septal defect, which is output when the subject's ECG potential data (specifically, a two-dimensional matrix where the vertical axis represents each lead (each of the 12 leads) and the horizontal axis represents time, i.e., a matrix showing the potential value at each time point for each lead) is input into the estimation model, and the correct data (i.e., information indicating whether the subject has atrial septal defect, acquired by the learning data acquisition unit 201).

The estimation model memory unit 204 stores the estimation model generated by the machine learning unit 203.

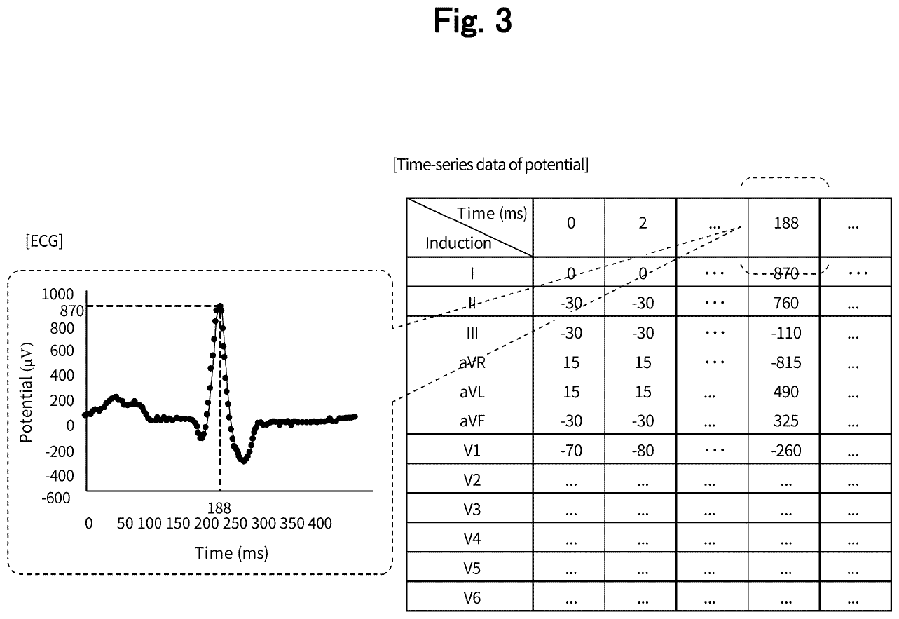

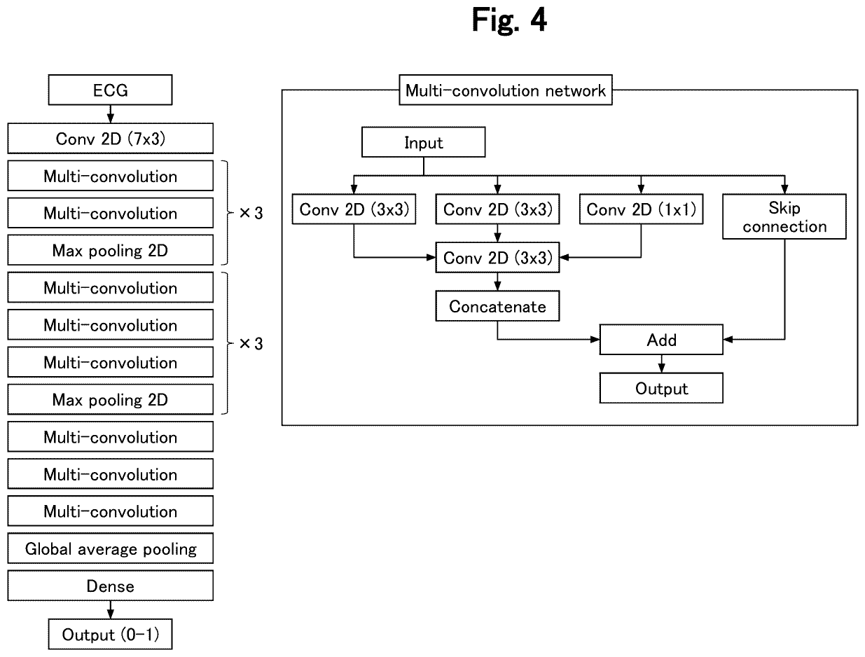

The estimation model will now be explained in detail with reference to FIGS. 3 to 4.

FIG. 3 is a diagram for explaining the time-series data of the underlying potential (ECG potential data) pertaining to an embodiment of the present invention. As shown in the [ECG] section of FIG. 3, an ECG graphically represents the potential (μV) at each time point (ms). In other words, an ECG is based on time-series data of potential (specifically, data where the potential value at each time point is recorded numerically). For example, the time-series data of the underlying potential for the ECG is the time-series data of the potential for each lead (i.e., the potential (μV) at each time point (ms)), as shown in [Time-Series Data of Potential] in FIG. 3. Note that the measurement interval for the potential varies depending on the ECG machine model, typically being 2 ms to 4 ms, but some models have an interval of 0.1 ms.

The present invention estimates whether atrial septal defect is present based on the time-series data of the underlying electrical potential of the ECG. In one embodiment of the present invention, the time-series data of the underlying electrical potential of the ECG can be represented as a two-dimensional matrix (i.e., a matrix showing the potential values at each time point for each lead), where the vertical axis represents each lead (each of the 12 leads) and the horizontal axis represents time. Specifically, where time is denoted as t,
the potential at t=1 for I, the potential at t=2 for I, . . .
the potential of II at t=1, the potential of II at t=2, . . . ,
the potential of III at t=1, the potential of III at t=2, . . .
aVR's potential at t=1, aVR's potential at t=2,
aVL at t=1, aVL at t=2, . . .
aVF potential at t=1, aVF potential at t=2, . . .
V1 at t=1, V2 at t=2, . . .
V2 at t=1, V2 at t=2, . . .
Potential at t=1 for V3, potential at t=2 for V3, . . .
Potential at t=1 for V4, potential at t=2 for V4, . . .
V5's potential at t=1, V5's potential at t=2, . . .
V6 at t=1, V6 at t=2, . . .

A two-dimensional matrix represented by these values can be used.

In one embodiment of the present invention, a Convolutional Neural Network (CNN) performs convolution on the time-series data of the underlying potentials of the ECG (specifically, a two-dimensional matrix where the vertical axis represents each lead (each of the 12 leads) and the horizontal axis represents time, i.e., a matrix showing the potential values at each time point for each lead) using a two-dimensional matrix called a filter or kernel. This enables the detection of complex information contained within the ECG.

Thus, in one embodiment of the present invention, since the potential data of the ECG (specifically, a two-dimensional matrix where the vertical axis represents each lead (each of the 12 leads) and the horizontal axis represents time, i.e., a matrix showing the potential value at each time point for each lead) is used, problems such as overlapping waves that occur when using waveforms are avoided. Furthermore, in one embodiment of the present invention, since convolution is performed on the ECG potential data (specifically, a two-dimensional matrix where the vertical axis represents each lead (each of the 12 leads) and the horizontal axis represents time, i.e., a matrix showing the potential values at each time point for each lead) (using a convolutional neural network), information spanning multiple leads, including differences between adjacent leads, can be extracted.

FIG. 4 is a diagram illustrating an estimation model according to an embodiment of the present invention. For example, the estimation model comprises an architecture, such as that shown in FIG. 4, and parameters determined by learning. The multi-layer convolutional network is constructed based on 2D (two-dimensional) convolution. The estimation model is trained multiple times using the multi-layer convolutional network.

In one embodiment of the present invention, configuring the neural network solely as a Convolutional Neural Network (CNN) enables computation parallelization and high-speed processing. Since the input data is ECG potential data, the system can handle any ECG regardless of vendor or model, and is unaffected by variations associated with imaging, such as calibration or noise.

### [ECG Monitor]

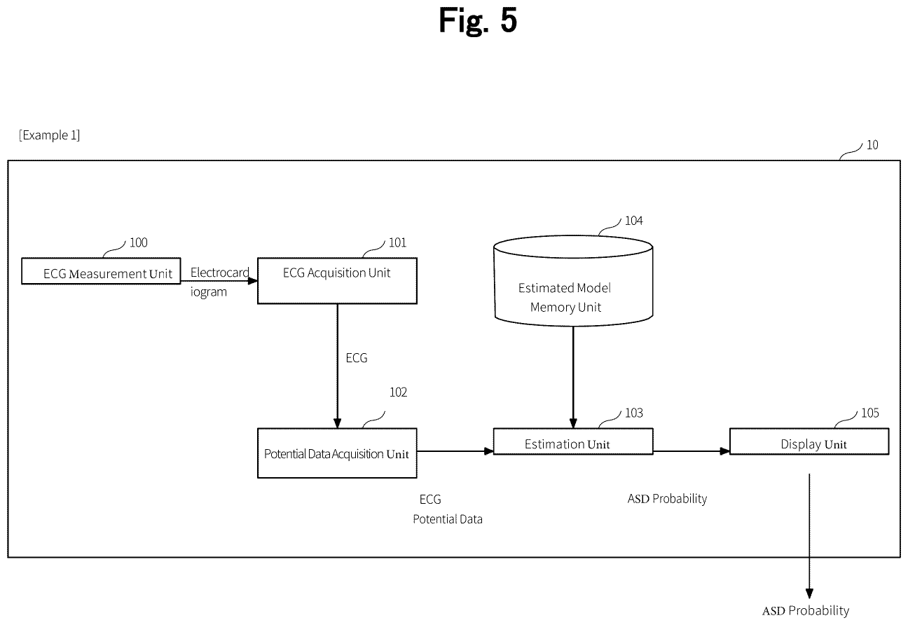

FIG. 5 is a functional block diagram of an ECG monitor 10 according to an embodiment of the present invention (Example 1). The ECG monitor 10 may include an ECG measurement unit 100, an ECG acquisition unit 101, a potential data acquisition unit 102, an estimation unit 103, an estimation model storage unit 104, and a display unit 105. By executing a program, the ECG monitor 10 can function as the ECG measurement unit 100, the ECG acquisition unit 101, the potential data acquisition unit 102, the estimation unit 103, and the display unit 105.

The ECG measurement unit 100 measures the patient's 30 ECG.

### [ECG]

As described above, the ECG is, for example, a 12-lead ECG. A 12-lead ECG uses four limb leads attached to the limbs and six chest leads attached to the chest. Six waveforms are derived from the limb leads, and one waveform is derived from each of the chest leads. Therefore, a total of twelve waveforms (I, II, III, aVR, aVL, aVF, V1, V2, V3, V4, V5, V6) are recorded.

The ECG acquisition unit 101 acquires the ECG of patient 30 measured by the ECG measurement unit 100.

The potential data acquisition unit 102 acquires the time-series data of the potential (ECG potential data) underlying the ECG of patient 30 from the ECG of patient 30 acquired by the ECG acquisition unit 101. Specifically, the potential data acquisition unit 102 generates a two-dimensional matrix (i.e., a matrix showing the potential values at each time for each lead) where the vertical axis represents each lead (each of the 12 leads) and the horizontal axis represents time.

The estimation unit 103 inputs the time-series data of the underlying electrical potentials (ECG potential data) for patient 30, obtained by the potential data acquisition unit 102 from the ECG of patient 30, into the estimation model stored in the estimation model storage unit 104, and outputs the probability that patient 30 has atrial septal defect. Specifically, estimation unit 103 inputs a two-dimensional matrix (i.e., a matrix showing the potential values at each time for each lead) into the estimation model, where the vertical axis represents each lead (each of the 12 leads) and the horizontal axis represents time, and outputs the probability that patient 30 has atrial septal defect.

The estimation model storage unit 104 stores an estimation model that estimates the probability that the subject has atrial septal defect from the subject's ECG potential data (specifically, a two-dimensional matrix where the vertical axis represents each lead (each of the 12 leads) and the horizontal axis represents time, i.e., a matrix showing the potential values at each time point for each lead).

The display unit 105 displays the probability estimated by the estimation unit 103 that the patient 30 has atrial septal defect (e.g., displayed together with the ECG measured by the ECG measurement unit 100).

### [ECG Management Device]

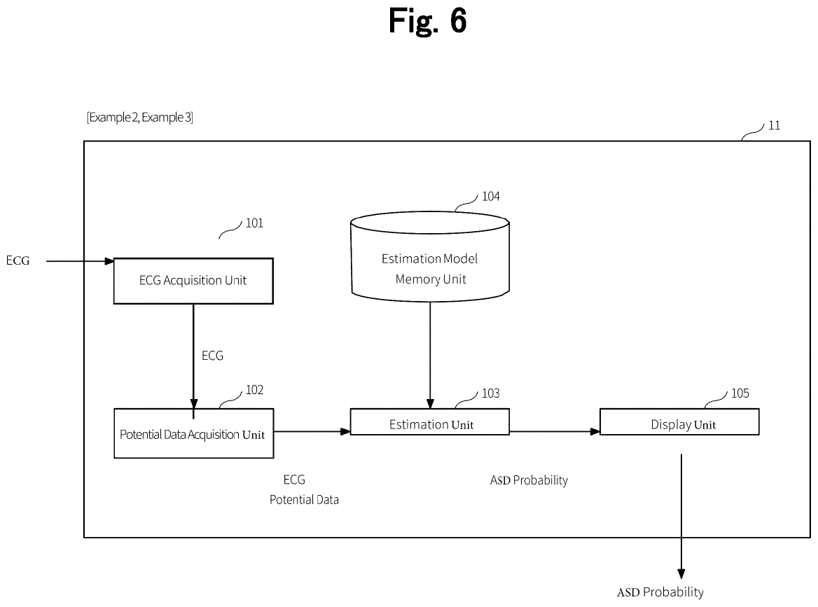

FIG. 6 is a functional block diagram of an ECG management device 11 according to an embodiment of the present invention (for Example 2 and Example 3). The ECG management device 11 may include an ECG acquisition unit 101, a potential data acquisition unit 102, an estimation unit 103, an estimation model storage unit 104, and a display unit 105. The ECG management device 11 can function as the ECG acquisition unit 101, the potential data acquisition unit 102, the estimation unit 103, and the display unit 105.

The ECG acquisition unit 101 acquires the ECG of the patient 30.

The potential data acquisition unit 102 is the same as the potential data acquisition unit 102 shown in FIG. 5, so its description is omitted.

The estimation unit 103 is the same as the estimation unit 103 in FIG. 5, so its description is omitted.

The estimation model storage unit 104 is the same as the estimation model storage unit 104 in FIG. 5, so its description is omitted.

The display unit 105 displays the probability estimated by the estimation unit 103 that the patient 30 has atrial septal defect (e.g., displayed together with the ECG measured by the ECG measurement unit 100). The display unit 105 may display this on the ECG management device 11, or it may display it on the ECG monitor 10 or another terminal.

### Processing Method

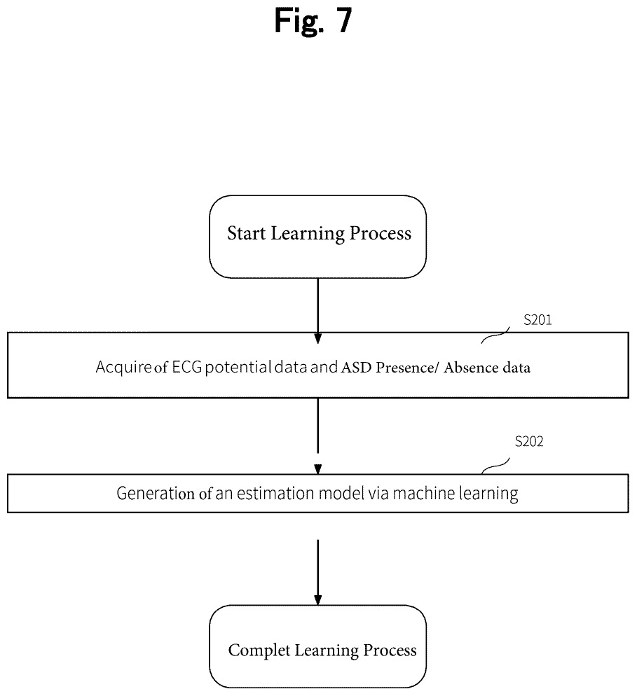

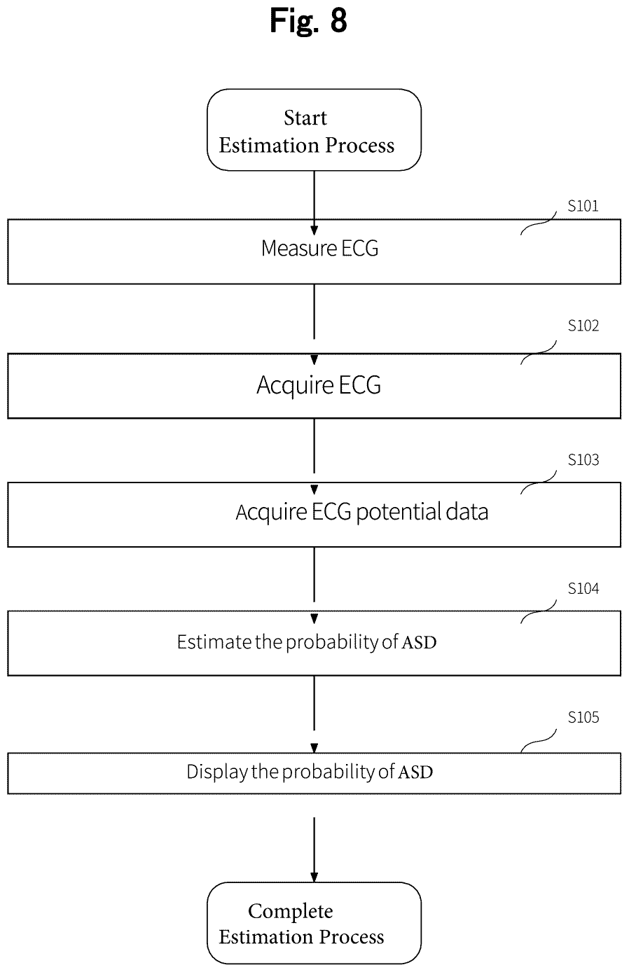

The following describes the learning processing method with reference to FIG. 7 and the estimation processing method with reference to FIG. 8.

### [Learning Process]

FIG. 7 is a flowchart of the learning process according to an embodiment of the present invention.

In step 201 (S201), the learning data acquisition unit 201 acquires paired data consisting of time-series data of the original potential (ECG potential data) forming the basis of the subject's electrocardiogram and information indicating whether or not the subject has atrial septal defect. As learning data, a two-dimensional matrix is used, where the vertical axis represents each lead (each of the 12 leads) and the horizontal axis represents time (i.e., a matrix showing the potential values at each time point for each lead).

In step 202 (S202), the machine learning unit 203 generates an estimation model through machine learning. Specifically, the machine learning unit 203 inputs the ECG potential data of the subject acquired in S201 (specifically, a two-dimensional matrix where the vertical axis represents each lead (each of the 12 leads) and the horizontal axis represents time, i.e., a matrix showing the potential values at each time point for each lead) into the estimation model and outputs whether the subject has an atrial septal defect. and the horizontal axis represents time, a two-dimensional matrix (i.e., a matrix showing the potential values at each time point for each lead) ) obtained in S201 into the estimation model. It then adjusts the parameters of the estimation model to minimize the error between the information indicating whether the subject has an atrial septal defect (ASD) and the correct answer data (i.e., the information indicating whether the subject has an ASD obtained in S201).

### [Estimation Processing]

FIG. 8 is a flowchart of the estimation process according to an embodiment of the present invention.

In step 101 (S101), the electrocardiogram (ECG) of patient 30 is measured. Note that the ECG may be one measured at any medical institution.

In step 102 (S102), the ECG acquisition unit 101 acquires the ECG of patient 30 measured in S101. The ECG acquisition unit 101 may acquire the ECG of patient 30 measured in real time by the ECG monitor 10, or it may acquire the ECG of patient 30 measured by the ECG monitor 10 in the past.

In step 103 (S103), the potential data acquisition unit 102 acquires the time-series data of the potential underlying the ECG of patient 30 (ECG potential data) from the ECG of patient 30 acquired in step S102. Specifically, the potential data acquisition unit 102 generates a two-dimensional matrix (i.e., a matrix showing the potential values at each time for each lead). The vertical axis represents each lead (each of the 12 leads), and the horizontal axis represents time.

In step 104 (S104), the estimation unit 103 inputs the time-series data of the underlying electrical potentials (ECG potential data) for patient 30, obtained from the ECG of patient 30 in step S103, into the estimation model (i.e., the estimation model generated in step S202 of FIG. 8) and outputs the probability that patient 30 has atrial septal defect. Specifically, estimation unit 103 inputs a two-dimensional matrix (i.e., a matrix showing the potential values at each time for each lead) into the estimation model, where the vertical axis represents each lead (each of the 12 leads) and the horizontal axis represents time, and outputs the probability that patient 30 has an atrial septal defect.

In step 105 (S105), the display unit 105 displays (e.g., displays together with the ECG) the probability that patient 30 has an atrial septal defect, as estimated in S104.

Physicians such as general practitioners or cardiologists can efficiently proceed to echocardiography, the definitive diagnostic test, by referring to the probability of atrial septal defect estimated using the present invention's method, based on ECGs obtained during examinations or health checkups.

### Display Example

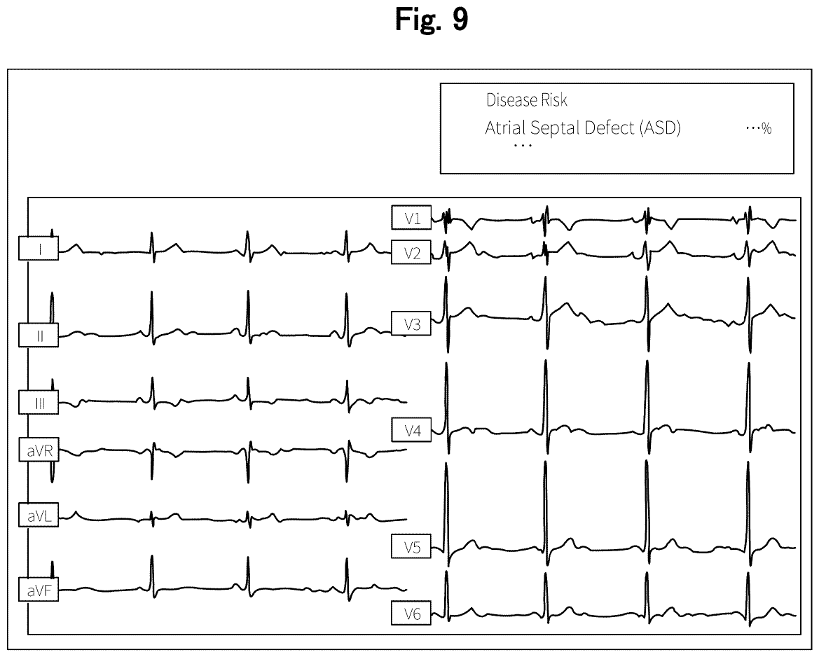

FIG. 9 shows an example of displaying an ECG and the probability that patient 30 has an atrial septal defect according to an embodiment of the present invention. For example, the ECG monitor 10, ECG management device 11, and other terminals display the ECG (e.g., a 12-lead ECG) and the probability that patient 30 has an atrial septal defect on a monitor. Alternatively, the ECG monitor 10, ECG management device 11, and other terminals may display the ECG (e.g., 12-lead ECG) and the probability that patient 30 has atrial septal defect by printing them on ECG recording paper.

### Estimation Model Accuracy

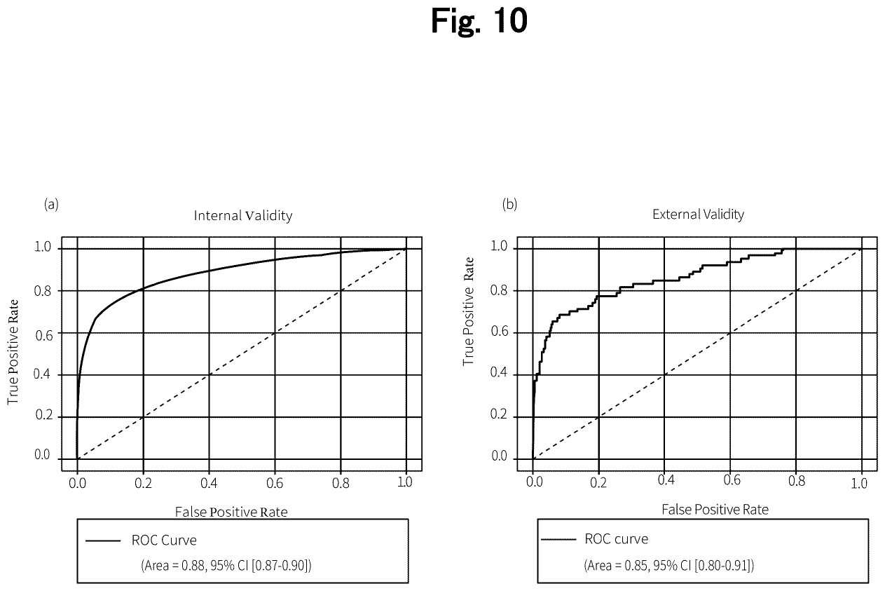

FIG. 10 is a diagram illustrating the accuracy of the estimation model according to an embodiment of the present invention. (a) shows the results of internal validation, and (b) shows the results of external validation.

### [(a) Internal Validation]

Internal validation shows the results of evaluating the estimation model using the same dataset that was used to generate the estimation model. The vertical axis represents the true positive rate (the proportion of actual positives correctly predicted as positive), and the horizontal axis represents the false positive rate (the proportion of actual negatives incorrectly predicted as positive). The area under the ROC curve (AUC) was 0.88. Furthermore, the 95% confidence interval for the AUC was 0.87 to 0.90.

### [b) External Validation]

External validity shows the results of evaluating the estimation model using a dataset different from the one used to generate the estimation model. The vertical axis represents the true positive rate (the proportion of actual positives correctly predicted as positive), and the horizontal axis represents the false positive rate (the proportion of actual negatives incorrectly predicted as positive). The area under the ROC curve (AUC) was 0.85. Furthermore, the 95% confidence interval for the AUC was 0.80-0.91.

Furthermore, the accuracy of the estimation model was evaluated as follows.
(Method) We recruited 7,177 patients aged 10-20 years who underwent 12-lead ECG testing at Keio University Hospital between January 2015 and March 2020, excluding those diagnosed with congenital heart disease other than ASD. We analyzed the ECG data obtained from these patients using the estimation model to evaluate its accuracy. For individuals with multiple ECGs, the maximum value of the prediction model output was used. ECGs acquired after ASD closure surgery were excluded.
(Results) Among the 7,177 individuals, 30 were ASD patients and 7,147 were non-ASD patients. The mean age was 15.9 years. Evaluation of the estimation model's accuracy in this cohort yielded an AUC (Area Under the Receiver Operating Characteristic curve): 0.89 (95% confidence interval: 0.82-0.97).

### Diagnosis

Traditionally, screening has been performed using classical methods such as auscultation, but sensitivity is extremely low during the asymptomatic phase. To detect ASD with high accuracy, tests like echocardiography are necessary. However, from the perspectives of time and cost, performing these tests on all individuals during health checkups as a screening method is difficult. Currently, no ECG changes specific to ASD are known, making ASD diagnosis via ECG impossible. However, by training AI on a large volume of ECGs, efficient ASD diagnosis was achieved (AUROC: 0.88, sensitivity 90%, specificity 90%).
Applying this invention to ECGs enables the highly accurate identification of cases strongly suspected of having ASD that would otherwise be missed. This facilitates early diagnosis via echocardiography and subsequent early intervention.
It should be noted that this invention is solely intended to assist the physician's judgment and does not eliminate the need for physician ECG interpretation.

Furthermore, it does not alter the application of ECG.

### Effect

ASD can cause cerebral infarction and heart failure, making early treatment desirable. However, conventional detection of ASD required time-consuming and costly examinations such as cardiac ultrasound. Since most ASD cases are asymptomatic, the likelihood of undergoing such examinations is low, resulting in many undetected cases. The present invention enables the detection of ASD from an electrocardiogram, which is a very simple and quick test that provides immediate results. This allows for the detection of previously undetected cases, leading to earlier treatment.

### Hardware Configuration

The hardware configuration is described below.

### [ECG Monitor]

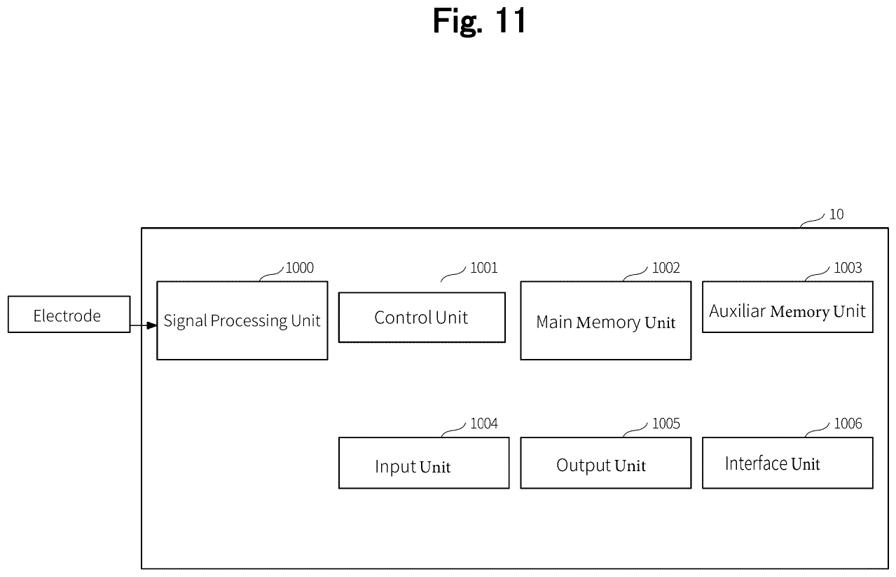

FIG. 11 is a hardware configuration diagram of the ECG monitor 10 according to an embodiment of the present invention. The ECG monitor 10 may include a signal processing unit 1000, a control unit 1001, a main memory unit 1002, an auxiliary memory unit 1003, an input unit 1004, an output unit 1005, and an interface unit 1006. Each of these is described below.

The signal processing unit 1000 includes an amplifier for amplifying signals acquired from electrodes and an A/D converter for digitizing the amplified signals.

The control unit 1001 is a processor (e.g., CPU (Central Processing Unit), GPU (Graphics Processing Unit), etc.) that executes various programs installed in the auxiliary storage unit 1003.

The main memory unit 1002 includes non-volatile memory (ROM (Read Only Memory) ) and volatile memory (RAM (Random Access Memory) ). The ROM stores various programs, data, etc., necessary for the control unit 1001 to execute the various programs installed in the auxiliary memory unit 1003. The RAM provides a working area that is deployed when various programs installed in the auxiliary storage unit 1003 are executed by the control unit 1001.

The auxiliary storage unit 1003 is an auxiliary storage device that stores various programs and information used when executing those programs.

Input unit 1004 is an input device through which the operator of electrocardiograph 10 inputs various instructions to electrocardiograph 10.

The output unit 1005 is an output device that outputs the internal state of the ECG monitor 10 and other information.

The interface unit 1006 is a communication device for connecting to a network and communicating with other devices.

### [Learning Device, ECG Management Device]

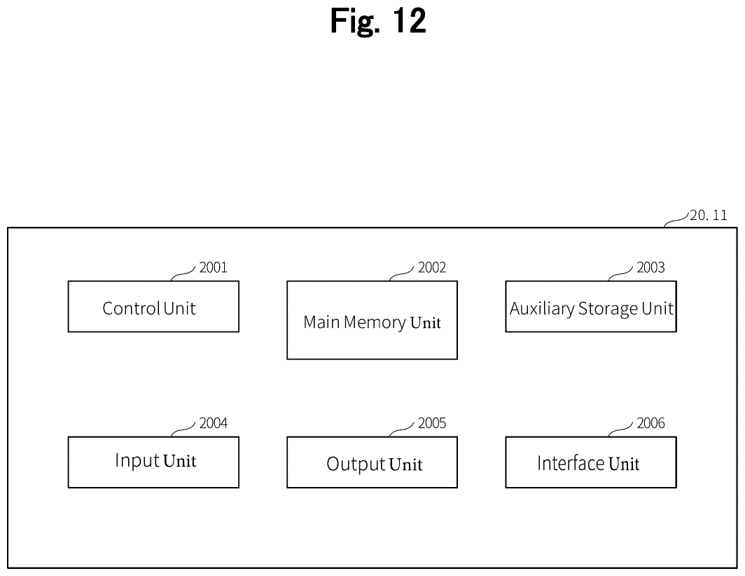

FIG. 12 is a hardware configuration diagram of the learning device 20 and ECG management device 11 according to an embodiment of the present invention. The learning device 20 and ECG management device 11 may include a control unit 2001, a main memory unit 2002, an auxiliary memory unit 2003, an input unit 2004, an output unit 2005, and an interface unit 2006. Each of these is described below.

The control unit 2001 is a processor (e.g., CPU (Central Processing Unit), GPU (Graphics Processing Unit), etc.) that executes various programs installed in the auxiliary storage unit 2003.

The main memory unit 2002 includes non-volatile memory (ROM (Read Only Memory)) and volatile memory (RAM (Random Access Memory)). The ROM stores various programs, data, etc., necessary for the control unit 2001 to execute the various programs installed in the auxiliary memory unit 2003. The RAM provides a working area that is expanded when various programs installed in the auxiliary storage unit 2003 are executed by the control unit 2001.

The auxiliary storage unit 2003 is an auxiliary storage device that stores various programs and information used when these programs are executed.

Input unit 2004 is an input device through which an operator of learning device 20 and ECG management device 11 inputs various instructions to learning device 20 and ECG management device 11.

The output unit 2005 is an output device that outputs the internal state and other information of the learning device 20 and the ECG management device 11.

The interface unit 2006 is a communication device for connecting to a network and communicating with other devices.

The present invention has been described in detail with reference to the above embodiments, but the invention is not limited to the specific embodiments described above. Various modifications and changes are possible within the scope of the essence of the invention as described in the claims.

This international application is based upon and claims priority to Japanese Patent Application No. 2023-060378 filed on Apr. 3, 2023, and the entire contents of which are incorporated herein by reference.

### DESCRIPTION OF REFERENCE NUMERALS

1. Electrocardiogram Management System
10 ECG Monitor
11 ECG management device
20 Learning Device
30. Patient
100 ECG Measurement Unit
101 ECG Acquisition Unit
102 Potential Data Acquisition Unit
103 Estimation Unit
104 Estimation Model Memory Unit
105 Display Unit
201 Learning Data Acquisition Unit
202 Learning Data Storage Unit
203 Machine Learning Unit
204 Estimated Model Storage Unit
1000 Signal Processing Unit
1001 Control Unit
1002 Main Memory Unit
1003 Auxiliary Memory Unit
1004 Input Unit
1005 Output Unit
1006 Interface Unit
2001 Control Unit
2002 Main Memory Unit
2003 Auxiliary Memory Unit
2004 Input Unit
2005 Output Unit
2006 Interface Unit

1. A non-transitory computer-readable recording medium storing a program for causing an estimation device to perform a method including:
acquiring an electrocardiogram (ECG) of a patient;
acquiring time-series data of a potential underlying the ECG of the patient from the ECG of the patient;
inputting the time-series data of the potential underlying the ECG of the patient into an estimation model that estimates a probability that a subject has an atrial septal defect based on time-series data of a potential underlying an ECG of the subject, and outputting a probability that the patient has the atrial septal defect; and
displaying the probability that the patient has the atrial septal defect.

2. The non-transitory computer-readable recording medium according to claim 1, wherein the time-series data of the potential is a two-dimensional matrix in which a vertical axis represents each lead and a horizontal axis represents time, indicating a potential value at each time point for the each lead.

3. The non-transitory computer-readable recording medium according to claim 1, wherein the ECG is a 12-lead ECG.

4. The non-transitory computer-readable recording medium according to claim 1, wherein in the displaying of the probability, the probability that the patient has the atrial septal defect is displayed along with the ECG of the patient.

5. The non-transitory computer-readable recording medium according to claim 1, wherein the estimation device is an electrocardiograph.

6. The non-transitory computer-readable recording medium according to claim 1, wherein the estimation device is a computer capable of acquiring the time-series data of the potential underlying the ECG.

7. An estimation device comprising:
a memory; and
a processor coupled to the memory and configured to:
an acquire an ECG of a patient;
acquire time-series data of a potential underlying the ECG of the patient from the ECG of the patient;
input the time-series data of the potential underlying the ECG of the patient into an estimation model that estimates a probability that a subject has an atrial septal defect based on time-series data of a potential underlying an ECG of the subject, and output a probability that the patient has the atrial septal defect; and
display the probability that the patient has the atrial septal defect.

8. A method performed by an estimation device, the method comprising:
acquiring an ECG of a patient;
acquiring time-series data of a potential underlying the ECG of the patient from the ECG of the patient;
inputting the time-series data of the potential underlying the ECG of the patient into an estimation model that estimates a probability that a subject has an atrial septal defect based on time-series data of a potential underlying an ECG of the subject, and outputting a probability that the patient has the atrial septal defect; and
displaying the probability that the patient has the atrial septal defect.

9. (canceled)

10. (canceled)
